(19) United States
(12) Patent Application Publication
(10) Pub. No.: US 2019/0267121 A1
(43) Pub. Date: Aug. 29, 2019

(54) MEDICAL RECOMMENDATION PLATFORM

(71) Applicant: Accenture Global Solutions Limited, Dublin (IE)

(72) Inventors: Paulo Victor de Sousa Moura, Recife (BR); Luiz Felipe Sampaio de Oliveira, Recife (BR)

(21) Appl. No.: 16/285,757

(22) Filed: Feb. 26, 2019

Related U.S. Application Data
(60) Provisional application No. 62/635,069, filed on Feb. 26, 2018.

(51) Int. Cl. G16H 20/10; G16H 50/20; G16H 10/60; G16H 20/60; G06N 20/00; G16H 50/50

(52) U.S. Cl. CPC G16H 20/10; G16H 50/20; G16H 10/60; G16H 50/30; G06N 20/00; G16H 50/50; G16H 20/60

(57) ABSTRACT

Methods, systems, and apparatus, including computer programs encoded on a computer storage medium, for a medical recommendation platform are disclosed. In one aspect, a method includes the actions of receiving an image of food and a blood glucose level of a user. The actions include providing the image of the food as an input to a food classification model to generate data indicating nutritional content of the food. The actions include receiving the data indicating the nutritional content of the food. The actions include providing the data indicating the nutritional content of the food and the blood glucose level of the user as an input to a medication recommendation model to generate data indicating an insulin dosage. The actions include receiving the data indicating the insulin dosage. The actions include providing, for output, instructions for the user to take the insulin dosage upon consuming the food.

### CROSS-REFERENCE TO RELATED APPLICATION

This application claims the benefit of U.S. application Ser. No. 62/635,069, filed Feb. 26, 2018, the contents of which are incorporated by reference

### TECHNICAL FIELD

This specification generally relates to machine learning.

### BACKGROUND

Machine learning is related to the study and construction of algorithms that can learn from and make predictions on data. The algorithms may overcome following strictly static program instructions by making data-driven predictions or decisions through building a model from sample inputs.

### SUMMARY

Managing diabetes may be challenging for a diabetic patient. A patient must learn how different foods affect the patient's blood glucose level and how the patient responds to insulin. The patient may use a software application that provides the patient the tools to allow the patient to more easily calculate appropriate insulin dosages. The software application may use machine learning to generate models that, when provided with features of an image of the food and the patient's medical history, may provide the patient with an appropriate insulin dosage before eating the food. The software application may continuously update the models based on the patient's actual blood glucose level after eating the food.

According to an innovative aspect of the subject matter described in this application, a method for a medical recommendation platform includes the actions of receiving, by a computing device, an image of food and a blood glucose level of a user; providing, by the computing device, the image of the food as an input to a food classification model that is configured to generate data indicating nutritional content of the food based on the image of the food; receiving, by the computing device and from the food classification model, the data indicating the nutritional content of the food; providing, by the computing device, the data indicating the nutritional content of the food and the blood glucose level of the user as an input to a medication recommendation model that is configured to generate data indicating an insulin dosage; receiving, by the computing device and from the medication recommendation model, the data indicating the insulin dosage; and providing, for output by the computing device, instructions for the user to take the insulin dosage upon consuming the food.

These and other implementations can each optionally include one or more of the following features. The actions further include receiving, by the computing device, training data that includes images of food and data indicating nutritional content of food in each image of food; and using machine learning, training, by the computing device, the food classification model using the images of food and the data indicating the nutritional content of the food in each image. The actions further include receiving, by the computing device, training data that includes images of food and data indicating food portions and food items included in each image of food; and using machine learning, training, by the computing device, a food identification model using the images of food and the data indicating the food portions and the food items included in each image of food. The action of providing the image of the food as an input to a food classification model that is configured to generate data indicating nutritional content of the food based on the image of the food includes providing the image of the food as an input to the food identification model that is configured to generate data indicating food items and food portions in the image of the food; and determining the nutritional content of the food based on the food items and the food portions in the image of the food.

The actions further include receiving, by the computing device, training data that includes nutritional content of consumed food, blood glucose levels of users before consuming the consumed food, blood glucose levels of the users after consuming the consumed food, and insulin dosages taken upon consuming the consumed food; and using machine learning, training, by the computing device, the medication recommendation model using the nutritional content of the consumed food, the blood glucose levels of the users before consuming the consumed food, the blood glucose levels of the users after eating the confused food, and the insulin dosages taken upon consuming the consumed food. The actions further include providing, by the computing device, the data indicating the nutritional content of the food, the blood glucose level of the user, and the insulin dosage as an input to a physical activities recommendation model that is configured to generate a suggested physical activity for the user; and providing, by the computing device for output, data indicating the suggested physical activity for the user.

The actions further include receiving, by the computing device, training data that includes nutritional content of consumed food, blood glucose levels of users before consuming the consumed food, blood glucose levels of the users after consuming the consumed food, insulin dosages taken by the users upon consuming the consumed food, and physical activities of the users; and using machine learning, training, by the computing device, the physical activities recommendation model using the nutritional content of the consumed food, the blood glucose levels of the users before consuming the consumed food, the blood glucose levels of the users after consuming the consumed food, the insulin dosages taken by the users upon consuming the consumed food, and the physical activities of the users. The actions further include receiving, by the computing device, an additional blood glucose level of the user; receiving, by the computing device, data indicating that the user consumed the food and the user took the insulin dosage; and using machine learning, updating, by the computing device, the food classification model using the additional blood glucose level of the user, the data indicating that the user consumed the food, and the data indicating that the user took the insulin dosage.

Other implementations of this aspect include corresponding systems, apparatus, and computer programs recorded on computer storage devices, each configured to perform the operations of the methods.

Particular implementations of the subject matter described in this specification can be implemented so as to realize one or more of the following advantages. A diabetic patient can use this technology to better manage blood glucose levels. A doctor can monitor the patient outside of the traditional doctor visit.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an example system for providing a diabetic patient recommendations to manage blood glucose levels.

FIG. 2 is a flowchart of an example process for providing a diabetic patient recommendations to manage blood glucose levels.

FIG. 3 is a flowchart of an example process for assisting a diabetic patient in managing blood glucose levels when consuming food.

FIG. 4 is an example of a computing device and a mobile computing device.

Like reference numbers and designations in the various drawings indicate like elements.

### DETAILED DESCRIPTION

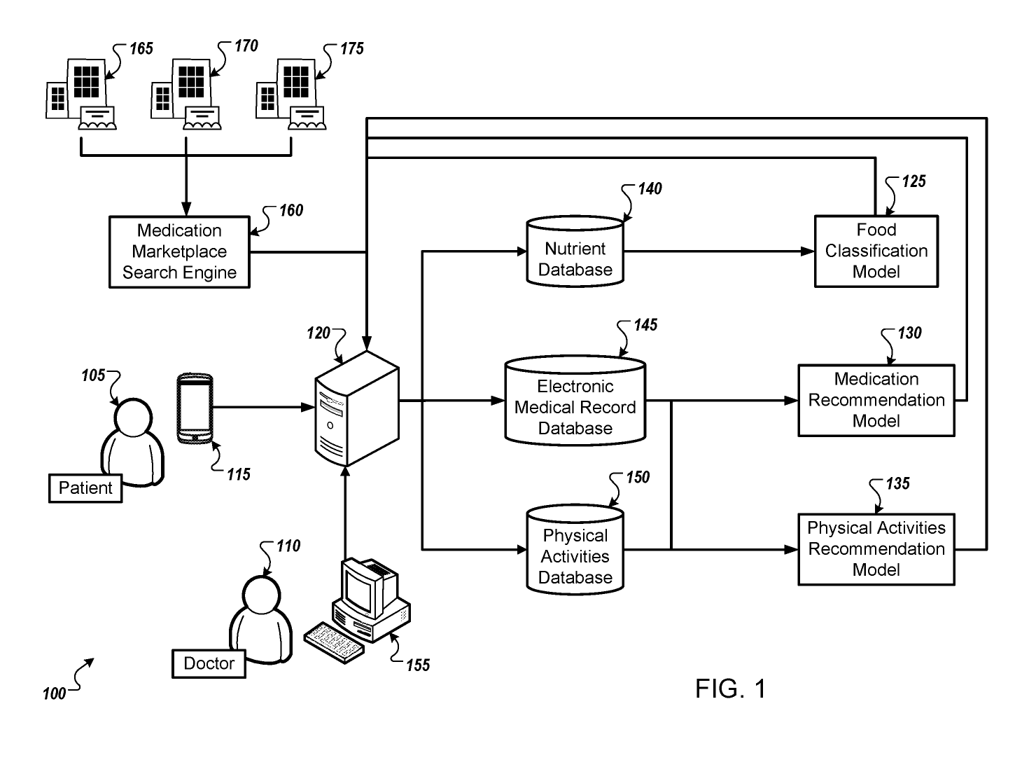

FIG. 1 is an example system 100 for providing a diabetic patient 105 recommendations to manage blood glucose levels. Briefly, and as described in more detail below, the system 100 collects data to train three different models using machine learning. The system 100 uses the models to provide a diabetic patient 105 with precise details regarding food that the patient 105 is about to eat, a recommendation regarding the amount of insulin to take before eating the food, and recommendations regarding physical activity. The patient's doctor 110 may monitor the patient 105 using the system 100, adjust any prescriptions as needed, and provide recommendations to the patient 105.

The system 100 is configured to provide the patient 105 with detailed information regarding the nutritional characteristics of the food. The patient may use mobile device 115 to capture an image of the food that the patient 105 is about to eat. The mobile device 115 may interact with the server 120, which applies a food classification model 125 to the image or features of the image. The food classification model 125 identifies the types of food in the picture and estimates the portion of each food item. Based on the type of food and the portion, the food classification model 125 generates data indicating the likely caloric content of the pictured food. For example, the patient 105 may capture an image of a plate of food that includes chicken, rice, and broccoli. The server 120 may apply the food classification model 125 to the features of the image and identify a grilled chicken breast that is about three hundred grams, a cup of cooked white rice, and a cup of broccoli with a tablespoon of butter. The food classification model 125 may determine that the chicken has two hundred calories, the rice has one hundred calories, and the broccoli with butter has one hundred fifty calories. The mobile device 115 may present the food caloric information to the patient 105.

The patient 105 may provide blood glucose information to the mobile device 115. The server 120 may apply the patient's blood glucose information and the estimated caloric content of the meal to the medication recommendation model 130.

The medication recommendation model 130 may be configured to provide a recommended insulin dosage for the patient 105 to take before eating the meal to manage the patient's blood glucose level. For example, the medication recommendation model 130 may recommend that the patient 105 take two units of insulin prior to eating the chicken, rice, and broccoli meal. The mobile device 115 may present the insulin dosage recommendation to the patient 105.

In some implementations, the medication recommendation model 130 may recommend an adjustment in the meal portion to help the patient 105 mange blood glucose levels. For example, the medication recommendation model 130 may recommend that the patient 105 only eat half a cup of the rice. The mobile device 115 may present the recommendation that the patient 105 only eat one half cup of rice. The patient 105 may accept or reject the recommendation. If the patient accepts or rejects the recommendation, then the medication recommendation model 130 may adjust the recommended insulin dosage as needed.

In some instances, the server 120 may provide recommendations regarding physical activity for the patient 105. The server 120 may apply the physical activity recommendation model 135 to current information related to the patient 105. The current information may include the patient's blood glucose level, the patient's gender, age, height, and weight, the patient's activity history, the patient's food consumption, and the patient's medication and insulin consumption. For example, the physical activity recommendation model 135 may recommend that the patient 105 walk one mile. The patient 105 may accept or reject the recommendation to walk one mile. In instances where the patient 105 rejects the recommendation, the physical activity recommendation model 135 may provide a recommendation for less physical activity that the patient 105 may be more likely to accept. In instances where the patient 105 accepts the recommendation, the server 120 may store the date, time, patient's blood glucose level, patient's food consumption, and/or activity performed.

The server 120 may use machine learning to train each of the food classification model 125, the medication recommendation model 130, and the physical activity recommendation model 135. The server 120 may collect training data for each model. In some implementations, the server 120 may continue to collect data to update each model. The updated model may be specific to the patient 105, general for all patients, or specific to a particular type of patient (e.g., women, age 30-39).

The server 120 may train the food classification model 125 using the nutrient database 140. The nutrient database 140 may include images of different foods and labels that identify the foods in each image and the amount of each food. Each image may include more than one type of food or only one type of food. The labels may identify the area of the image where the corresponding food is located. The nutrient database 140 may also have access to a caloric database that identifies the caloric content of each food and other nutritional information such as amount of carbohydrates, fat, protein, vitamins, and minerals.

The server 120 may train the food classification model 125 such that the food classification model 125 is configured to receive an image that includes a representation of food. The output of the food classification model 125 may be the foods included in the image and the portions of each food in the image. In some implementations, the output of the food classification model 125 may be the nutritional content of the food included in the image.

The server 120 may train the medication recommendation model 130 using the electronic medical record database 145. The electronic medical record database 145 may include training data collected from many different patients. Each patient record may be anonymized to ensure patient confidentiality. Each patient record may include a patient's gender, age, height, weight history, medical conditions, prescribed medications, and blood glucose level history. In some implementations, the patient record may include food consumption history and physical activity history. The server 120 may train the medication recommendation model 130 to recommend an appropriate dosage of insulin or prescribed medication at various points throughout the day, including in response to the patient 105 indicating that the patient 105 is about to eat food.

The server 120 may train the medication recommendation model 130 such that the medication recommendation model 130 is configured to receive data indicating the patient's gender, age, height, weight, blood glucose level, and nutritional content of food that the patient 105 is about to eat. The output of the medication recommendation model 130 may be a recommended insulin dosage that the patient 105 should take before eating the food.

The server 120 may train the physical activities recommendation model 135 using the physical activities database 150. The physical activities database 150 may be similar to the electronic medical record database 145 in that the physical activities database 150 may include training data collected from different patients. In some implementations, the electronic medical record database 145 includes only training data collected from the patient 105. The physical activities database 150 may be anonymized to ensure patient confidentiality. Each patient record may include a patient's gender, age, height, weight history, medical conditions, blood glucose history, and physical activity history. In some implementations, each patient record may include food consumption history and prescribed medications. The server 120 may train the physical activities recommendation model 135 to recommend an appropriate physical activity to the patient 105 to manage blood glucose levels.

The server 120 may train the physical activities recommendation model 135 such that the physical activities recommendation model 135 is configured to receive data indicating a patient's gender, age, height, weight, nutritional content of food that the patient 105 has eaten, blood glucose level before eating the food, and blood glucose level after eating the food. The output of the physical activities recommendation model 135 may be a suggested activity for the patient 105 such as walking a mile. In some implementations, the physical activities recommendation model 135 may be configured to receive data indicating any medications that the patient 105 is taking.

In some implementations, the server 120 may update the food classification model 125, the medication recommendation model 130, and the physical activities recommendation model 135 based on the feedback received from the patient 105. The patient may provide blood glucose level information to the server 120. The server 120 may use the blood glucose information and the information gathered from the patient's food consumption and activity history to update the models. For example, the server 120 may use the information of the patient's blood glucose level before and after eating a meal of chicken, rice, and broccoli and taking two units of insulin. The server 120 may use this information to update the medication recommendation model 130 and improve the accuracy of future insulin recommendations by retraining the medication recommendation model 130 using machine learning. In some implementations, the server 120 may update the models using information from the patient 105 so that the models become tailored to the patient 105. In some implementations, the server 120 may update the models using information from other patients, thus maintaining models that provide general recommendations.

The system 100 may include a terminal 155 for the doctor 110 to interact with the server 120. The terminal 155 may provide the doctor 110 with the ability to monitor the progress of the patient 105 remotely. This monitoring may supplement in person interactions between the patient 105 and the doctor 110. The doctor 110 may provide updates to the patient's prescriptions using the terminal 155. The server 105 may incorporate any updated prescriptions to the medication recommendation model 130.

The system 100 includes a medication marketplace search engine 160. The medication marketplace search engine is configured to search prices of various medication providers to search for the medication provider that is the lowest cost to the patient 105. For example, the medication marketplace search engine 160 may compare medication provider 165, medication provider 170, and medication provider 175 to identify the lowest cost provider of insulin for the patient 105. The server 120 may automatically order the medication as needed by the patient 105 based on the doctor's prescription, the medication recommendation model 130, and the patient's indications regarding how much medicine the patient 105 took.

In some implementations, the server 120 may format data received by the patient 105 via computing device 115, data received by the doctor 110 via computing device 155, data received from the food classification model 125, data received from the medication recommendation model 130, data received from the physical activities recommendation model 135, and/or the medication marketplace search engine 160 so that the formatted data can be presented to the patient 105 via computing device 115 and/or the doctor 110 via computing device 155. For example, the patient 105 may view data through an application running on the computing device 115. The physical activities recommendation model 135 may output an activity recommendation in a format that is not presentable on the computing device 115. The server 120 may format the activity recommendation and present the activity recommendation on the application of the computing device 115. As another example, the medication recommendation model 130 may recommend an insulin dosage for the user 105 to take in conjunction with a meal. Upon receiving an indication that the user 105 accepted the recommendation and took the recommended insulin dosage, the server 120 may format and store a record of the insulin dosage in the electronic medical record database 145. The server 120 may reformat the record of the insulin dosage when presenting the record of the insulin dosage on the computing device 115 and the computing device 155.

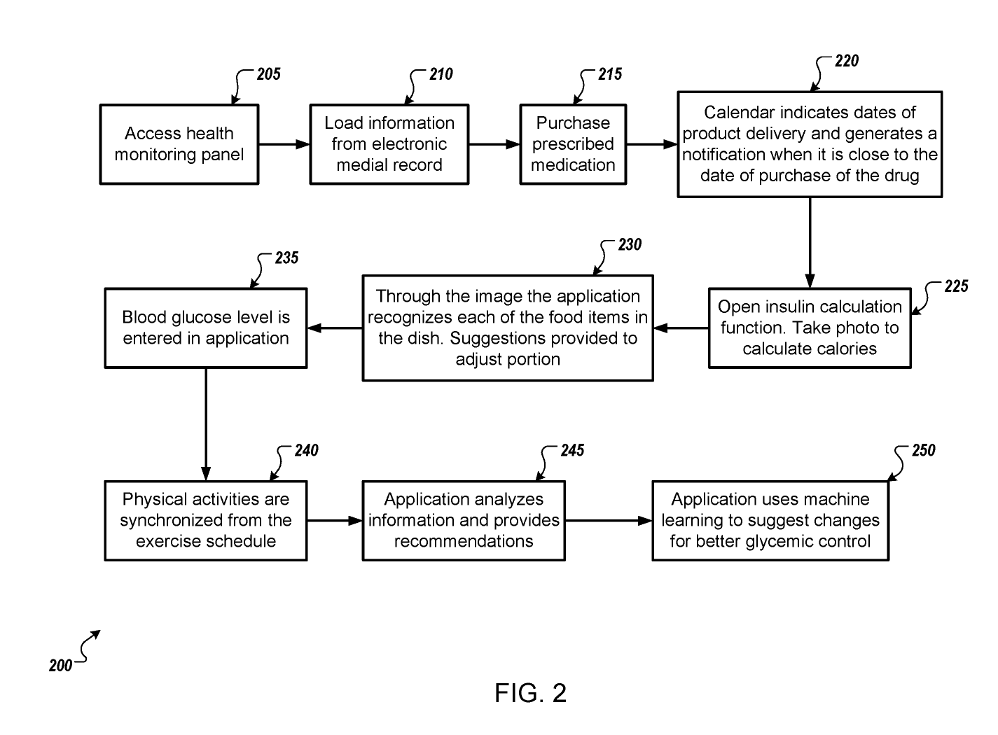

FIG. 2 is a flowchart of an example process 200 for providing a diabetic patient recommendations to manage blood glucose levels. In general the process 200 uses machine learning to generate models that provide a diabetic patient recommended insulin dosages to manage blood glucose levels. The process 200 will be described as being performed by a computer system comprising one or more computers, for example, the system 100 of FIG. 1.

The patient accesses the system using an application that includes a health monitoring panel and that is running on, for example, a mobile device (205). The system may load information from the patient's electronic medical record (210). The electronic medical record may include the patient's prescriptions. The system may automatically purchase the prescribed medications (215). In some implementations, the system may search different pharmaceutical providers to find the provider that can deliver the prescription at the needed time and at the lowest cost. In some implementations, the system may make suggestions for other products that may be related to the prescribed medications. In some implementations, the system may use a subscription service to purchase the prescribed medications.

The system may update the patient's calendar to indicate when the medications will likely arrive (220). The patient may receive a notification to remind the patient that the delivery date is approaching. The patient may use the patient's mobile device to take a picture of any food the patient is about to eat. The system may receive the photo of the food and apply features of the photo or a representation of the photo to a model to estimate the calories and other nutrition of the food (225). The system may recommend that the patient adjust the portions (230), by for example, only eating half of the pasta that the patient captured in the photo. The patient may accept or reject the portion recommendation. The system may recommend an insulin dosage for the food.

The system receives a blood glucose from the patient before and after eating the food (235). The system may recommend physical activities, and the patient may enter the patient's physical activities into the system (240). The system may analyze the patient's food consumption, insulin usage, physical activities and provide a recommendation for physical activities, insulin usage, and/or food consumption (245). The system may use machine learning to tailor the recommendations to the patient to improve the patient's ability to control the patient's glucose level (250).

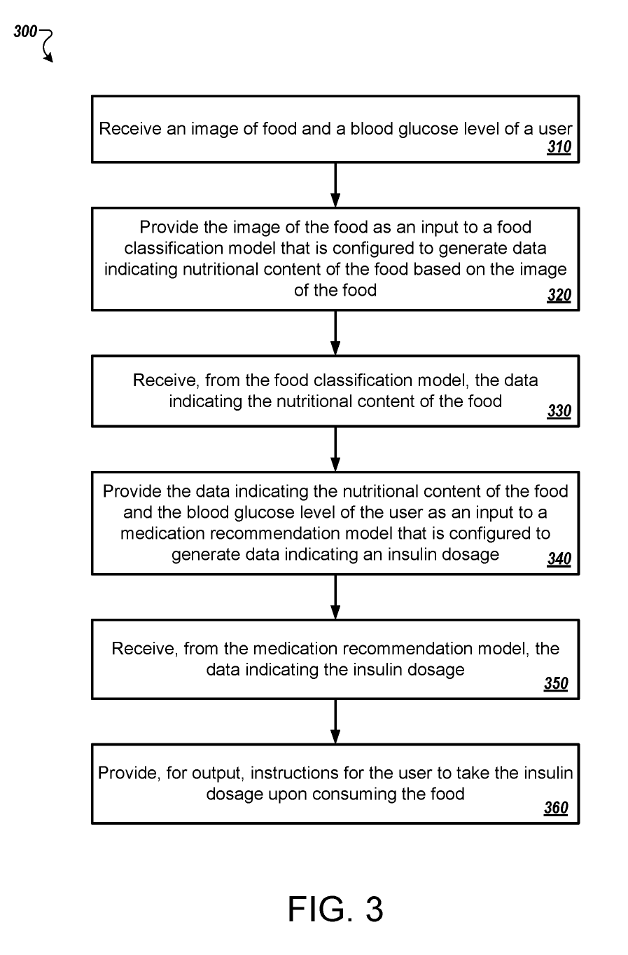

FIG. 3 is a flowchart of an example process 300 for assisting a diabetic patient in managing blood glucose levels when consuming food. In general, the process 300 uses models trained using machine learning to analyze the actions of a diabetic patient and provide recommended actions for the patient. The recommended actions may include a recommended insulin dosage and/or a recommended activity. The process 300 will be described as being performed by a computer system comprising one or more computers, for example, the system 100 of FIG. 1.

The system receives an image of food and a blood glucose level of a user (310). In some implementations, the user takes a picture of the food that the user is about to eat. The user may also input the user's current blood glucose level into the system. In some implementations, the system may include a blood glucose monitoring device or be in communication with a blood glucose monitoring device.

The system provides the image of the food as an input to a food classification model that is configured to generate data indicating nutritional content of the food based on the image of the food (320). In some implementations, the system includes the food classification model. In some implementations, the system accesses another system that includes the food classification model.

In some implementations, the system trains the food classification model using machine learning techniques. The system may use training data that includes images of food and the nutritional content of the food in each image. Some images may include only one food item. Some images may include more than one food item. In instances where the image includes more than one food item, the nutritional content of the food in each image may include the nutritional content of each food item in an image. The system may train, using machine learning, the food classification model to receive an image of food and output the nutritional content of the food in the received image. The food classification model may identify the food items in each received image and the nutritional content of each food item.

In some implementations, the system may retrain, using machine learning, the food classification model as the system receives additional images of food and the nutritional content of the food in each image. The additional images of food may be images that the food classification model has processed, and the system may receive data indicating the nutritional content of the food as determined from another method. For example, a user may determine the nutritional content by examining labels and cooking methods of the food in the image and provide the nutritional content to the system to retrain food classification model.

In some implementations, the training data includes images of food and the food items and portions in each image. Some images may include only one food item. Some images may include more than one food item. The system may train, using machine learning, the food classification model to receive an image of food and output the portion of each food item in the image.

In some implementations, the system may retrain, using machine learning, the food classification model as the system receives additional images of food and the portion of each food item in the received image. The additional images of food may be images that the food classification model has processed, and the system may receive data indicating portion of each food item and the type of each food item in the image. The system may receive the data identifying the food item and the type of food item from a user who measures each food item and identifies the food item.

The system receives, from the food classification model, the data indicating the nutritional content of the food (330). In some implementations, the food classification model outputs the nutritional content of the food directly. In some implementations, the food classification model outputs the food items and the portions of the food items in the image. The system then accesses a nutrition database to determine each food item's nutritional content.

The system provides the data indicating the nutritional content of the food and the blood glucose level of the user as an input to a medication recommendation model that is configured to generate data indicating an insulin dosage (340). In some implementations, the system includes the medication recommendation model. In some implementations, the system accesses another system that includes the medication recommendation model.

In some implementations, the system trains the medication recommendation model using machine learning techniques. The system may use training data that includes nutritional content of consumed food, blood glucose levels of users before consuming the consumed food, blood glucose levels of the users after consuming the consumed food, and insulin dosages taken upon consuming the consumed food. The system may train, using machine learning, the medication recommendation model to receive the blood glucose level of the user and the nutritional content of the food that the user is about to eat. The medication recommendation model outputs a recommended insulin dosage for the user to take in conjunction with eating the food.

In some implementations, the system receives the blood glucose level of the user after eating the food and taking the recommended insulin dosage. The system may use the blood glucose level of the user after eating the food to retrain the medication recommendation model. In some implementations, the system may retrain the medication recommendation model for the user such that the system uses the retrained medication recommendation model to recommend insulin dosages only for the user and not other users. In some implementations, the system may retrain the medication recommendation model for the user and similar users based on blood glucose levels after eating food. The similar users may be users in the same demographic as the user. In some implementations, the system retrains the medication recommendation model for all users and using blood glucose level feedback from all users.

The system receives, from the medication recommendation model, the data indicating the insulin dosage (350). The system provides, for output, instructions for the user to take the insulin dosage upon consuming the food (360). In some implementations, the system outputs the data indicating the insulin dosage recommendation directly to the system for viewing on the screen of the system. In some implementations, the system outputs the data indicating the insulin dosage recommendation to an automated insulin pump.

In some implementations, the system provides the data indicating the nutritional content of the food, the blood glucose level of the user, and the recommended insulin dosage as an input to a physical activities recommendation model. The physical activities recommendation model may be configured to output a suggested physical activity for the user. In some implementations, the user may accept the activity suggestion and provide an indication to the system that the user performed the activity. The user may provide additional blood glucose level data before and after the completion of the activity if the user did not already provide the blood glucose level data previously. In some implementations, the user may reject the activity suggestion. In this instance, the physical activities recommendation model may suggest an alternative activity. The physical activities recommendation model may continue to suggest alternative activities until the user accepts the activity.

In some implementations, the system trains, using machine learning, the physical activities recommendation model. The training data may include nutritional content of consumed food, blood glucose levels of users before consuming the consumed food, blood glucose levels of the users after consuming the consumed food, insulin dosages taken by the users upon consuming the consumed food, physical activities of the users, and/or blood glucose levels after performing the activity. In some implementations, the training data may also include demographic information such as gender and age of each user. The training data may also include the height and weight of each user.

The system may train the physical activities recommendation model to receive blood glucose level of a user, height of the user, and the weight of the user. The physical activities recommendation model may be configured to output a suggested activity. The system may train the physical activities recommendation model to accept different inputs. For example, the system may train the physical activities recommendation model to accept the nutritional content of consumed food, the blood glucose levels of the user before consuming the food, and the blood glucose level of the user after consuming the food. In this instance, the physical activities recommendation model may also output a recommended physical activity.

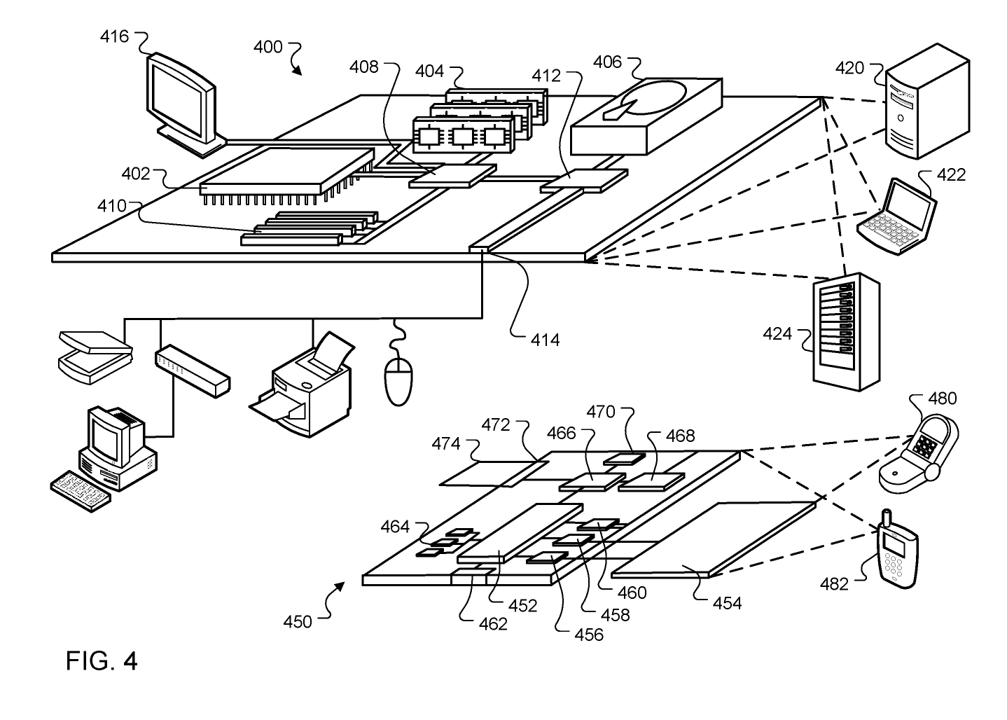

FIG. 4 shows an example of a computing device 400 and a mobile computing device 450 that can be used to implement the techniques described here. The computing device 400 is intended to represent various forms of digital computers, such as laptops, desktops, workstations, personal digital assistants, servers, blade servers, mainframes, and other appropriate computers. The mobile computing device 450 is intended to represent various forms of mobile devices, such as personal digital assistants, cellular telephones, smart-phones, wireless (e.g., Bluetooth) headsets, hearing aid, smart watches, smart glasses, activity trackers, and other similar computing devices. The components shown here, their connections and relationships, and their functions, are meant to be examples only, and are not meant to be limiting.

The computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and may be mounted on a common motherboard or in other manners as appropriate. The processor 402 can process instructions for execution within the computing device 400, including instructions stored in the memory 404 or on the storage device 406 to display graphical information for a GUI on an external input/output device, such as a display 416 coupled to the high-speed interface 408. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 404 stores information within the computing device 400. In some implementations, the memory 404 is a volatile memory unit or units. In some implementations, the memory 404 is a non-volatile memory unit or units. The memory 404 may also be another form of computer-readable medium, such as a magnetic or optical disk.

The storage device 406 is capable of providing mass storage for the computing device 400. In some implementations, the storage device 406 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 402), perform one or more methods, such as those described above. The instructions can also be stored by one or more storage devices such as computer- or machine-readable mediums (for example, the memory 404, the storage device 406, or memory on the processor 402).

The high-speed interface 408 manages bandwidth-intensive operations for the computing device 400, while the low-speed interface 412 manages lower bandwidth-intensive operations. Such allocation of functions is an example only. In some implementations, the high-speed interface 408 is coupled to the memory 404, the display 416 (e.g., through a graphics processor or accelerator), and to the high-speed expansion ports 410, which may accept various expansion cards (not shown). In the implementation, the low-speed interface 412 is coupled to the storage device 406 and the low-speed expansion port 414. The low-speed expansion port 414, which may include various communication ports (e.g., USB, Bluetooth, Ethernet, wireless Ethernet) may be coupled to one or more input/output devices, such as a keyboard, a pointing device, a scanner, a microphone, speakers, or a networking device such as a switch or router, e.g., through a network adapter.

The computing device 400 may be implemented in a number of different forms, as shown in the figure. For example, it may be implemented as a standard server 420, or multiple times in a group of such servers. In addition, it may be implemented in a personal computer such as a laptop computer 422. It may also be implemented as part of a rack server system 424. Alternatively, components from the computing device 400 may be combined with other components in a mobile device (not shown), such as a mobile computing device 450. Each of such devices may contain one or more of the computing device 400 and the mobile computing device 450, and an entire system may be made up of multiple computing devices communicating with each other.

The mobile computing device 450 includes a processor 452, a memory 464, an input/output device such as a touch-enabled display 454, a communication interface 466, and a transceiver 468, among other components. The mobile computing device 450 may also be provided with a storage device, such as a micro-drive or other device, to provide additional storage. Each of the processor 452, the memory 464, the display 454, the communication interface 466, and the transceiver 468, are interconnected using various buses, and several of the components may be mounted on a common motherboard or in other manners as appropriate.

The processor 452 can execute instructions within the mobile computing device 450, including instructions stored in the memory 464. The processor 452 may be implemented as a chipset of chips that include separate and multiple analog and digital processors. The processor 452 may provide, for example, for coordination of the other components of the mobile computing device 450, such as control of user interfaces, applications run by the mobile computing device 450, and wireless communication by the mobile computing device 450.

The processor 452 may communicate with a user through a control interface 458 and a display interface 456 coupled to the display 454. The display 454 may be, for example, a TFT (Thin-Film-Transistor Liquid Crystal Display) display or an OLED (Organic Light Emitting Diode) display, or other appropriate display technology. The display interface 456 may comprise appropriate circuitry for driving the display 454 to present graphical and other information to a user. The control interface 458 may receive commands from a user and convert them for submission to the processor 452. In addition, an external interface 462 may provide communication with the processor 452, so as to enable near area communication of the mobile computing device 450 with other devices. The external interface 462 may provide, for example, for wired communication in some implementations, or for wireless communication in other implementations, and multiple interfaces may also be used.

The memory 464 stores information within the mobile computing device 450. The memory 464 can be implemented as one or more of a computer-readable medium or media, a volatile memory unit or units, or a non-volatile memory unit or units. An expansion memory 474 may also be provided and connected to the mobile computing device 450 through an expansion interface 472, which may include, for example, a SIMM (Single In Line Memory Module) card interface. The expansion memory 474 may provide extra storage space for the mobile computing device 450, or may also store applications or other information for the mobile computing device 450. Specifically, the expansion memory 474 may include instructions to carry out or supplement the processes described above, and may include secure information also. Thus, for example, the expansion memory 474 may be provided as a security module for the mobile computing device 450, and may be programmed with instructions that permit secure use of the mobile computing device 450. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. that the instructions, when executed by one or more processing devices (for example, processor 452), perform one or more methods, such as those described above. The instructions can also be stored by one or more storage devices, such as one or more computer- or machine-readable mediums (for example, the memory 464, the expansion memory 474, or memory on the processor 452). In some implementations, the instructions can be received in a propagated signal, for example, over the transceiver 468 or the external interface 462.

The mobile computing device 450 may communicate wirelessly through the communication interface 466, which may include digital signal processing circuitry where necessary. The communication interface 466 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 468 using a radio-frequency. In addition, short-range communication may occur, such as using a Bluetooth, WiFi, or other such transceiver (not shown). In addition, a GPS (Global Positioning System) receiver module 470 may provide additional navigation- and location-related wireless data to the mobile computing device 450, which may be used as appropriate by applications running on the mobile computing device 450.

The mobile computing device 450 may also communicate audibly using an audio codec 460, which may receive spoken information from a user and convert it to usable digital information. The audio codec 460 may likewise generate audible sound for a user, such as through a speaker, e.g., in a handset of the mobile computing device 450. Such sound may include sound from voice telephone calls, may include recorded sound (e.g., voice messages, music files, etc.) and may also include sound generated by applications operating on the mobile computing device 450.

The mobile computing device 450 may be implemented in a number of different forms, as shown in the figure. For example, it may be implemented as a cellular telephone 480. It may also be implemented as part of a smart-phone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. For example, while a client application is described as accessing the delegate(s), in other implementations the delegate(s) may be employed by other applications implemented by one or more processors, such as an application executing on one or more servers. In addition, the logic flows depicted in the figures do not require the particular order shown, or sequential order, to achieve desirable results. In addition, other actions may be provided, or actions may be eliminated, from the described flows, and other components may be added to, or removed from, the described systems. Accordingly, other implementations are within the scope of the following claims.

What is claimed is:

1. A computer-implemented method, comprising:
receiving, by a computing device, an image of food and a blood glucose level of a user;
providing, by the computing device, the image of the food as an input to a food classification model that is configured to generate data indicating nutritional content of the food based on the image of the food;
receiving, by the computing device and from the food classification model, the data indicating the nutritional content of the food;
providing, by the computing device, the data indicating the nutritional content of the food and the blood glucose level of the user as an input to a medication recommendation model that is configured to generate data indicating an insulin dosage;
receiving, by the computing device and from the medication recommendation model, the data indicating the insulin dosage; and
providing, for output by the computing device, instructions for the user to take the insulin dosage upon consuming the food.

2. The method of claim 1, comprising:
receiving, by the computing device, training data that includes images of food and data indicating nutritional content of food in each image of food; and
using machine learning, training, by the computing device, the food classification model using the images of food and the data indicating the nutritional content of the food in each image.

3. The method of claim 1, comprising:
receiving, by the computing device, training data that includes images of food and data indicating food portions and food items included in each image of food; and
using machine learning, training, by the computing device, a food identification model using the images of food and the data indicating the food portions and the food items included in each image of food.

4. The method of claim 1, wherein providing the image of the food as an input to a food classification model that is configured to generate data indicating nutritional content of the food based on the image of the food comprises:
providing the image of the food as an input to the food identification model that is configured to generate data indicating food items and food portions in the image of the food; and
determining the nutritional content of the food based on the food items and the food portions in the image of the food.

5. The method of claim 1, comprising:
receiving, by the computing device, training data that includes nutritional content of consumed food, blood glucose levels of users before consuming the consumed food, blood glucose levels of the users after consuming the consumed food, and insulin dosages taken upon consuming the consumed food; and
using machine learning, training, by the computing device, the medication recommendation model using the nutritional content of the consumed food, the blood glucose levels of the users before consuming the consumed food, the blood glucose levels of the users after eating the confused food, and the insulin dosages taken upon consuming the consumed food.

6. The method of claim 1, comprising:
providing, by the computing device, the data indicating the nutritional content of the food, the blood glucose level of the user, and the insulin dosage as an input to a physical activities recommendation model that is configured to generate a suggested physical activity for the user; and
providing, by the computing device for output, data indicating the suggested physical activity for the user.

7. The method of claim 6, comprising:
receiving, by the computing device, training data that includes nutritional content of consumed food, blood glucose levels of users before consuming the consumed food, blood glucose levels of the users after consuming the consumed food, insulin dosages taken by the users upon consuming the consumed food, and physical activities of the users; and
using machine learning, training, by the computing device, the physical activities recommendation model using the nutritional content of the consumed food, the blood glucose levels of the users before consuming the consumed food, the blood glucose levels of the users after consuming the consumed food, the insulin dosages taken by the users upon consuming the consumed food, and the physical activities of the users.

8. The method of claim 1, comprising:
receiving, by the computing device, an additional blood glucose level of the user;
receiving, by the computing device, data indicating that the user consumed the food and the user took the insulin dosage; and
using machine learning, updating, by the computing device, the food classification model using the additional blood glucose level of the user, the data indicating that the user consumed the food, and the data indicating that the user took the insulin dosage.

9. A system comprising:
one or more computers; and
one or more storage devices storing instructions that are operable, when executed by the one or more computers, to cause the one or more computers to perform operations comprising:
receiving, by a computing device, an image of food and a blood glucose level of a user;
providing, by the computing device, the image of the food as an input to a food classification model that is configured to generate data indicating nutritional content of the food based on the image of the food;
receiving, by the computing device and from the food classification model, the data indicating the nutritional content of the food;
providing, by the computing device, the data indicating the nutritional content of the food and the blood glucose level of the user as an input to a medication recommendation model that is configured to generate data indicating an insulin dosage;
receiving, by the computing device and from the medication recommendation model, the data indicating the insulin dosage; and
providing, for output by the computing device, instructions for the user to take the insulin dosage upon consuming the food.

10. The system of claim 9, wherein the operations comprise:
receiving, by the computing device, training data that includes images of food and data indicating nutritional content of food in each image of food; and
using machine learning, training, by the computing device, the food classification model using the images of food and the data indicating the nutritional content of the food in each image.

11. The system of claim 9, wherein the operations comprise:
receiving, by the computing device, training data that includes images of food and data indicating food portions and food items included in each image of food; and
using machine learning, training, by the computing device, a food identification model using the images of food and the data indicating the food portions and the food items included in each image of food.

12. The system of claim 9, wherein providing the image of the food as an input to a food classification model that is configured to generate data indicating nutritional content of the food based on the image of the food comprises:
providing the image of the food as an input to the food identification model that is configured to generate data indicating food items and food portions in the image of the food; and
determining the nutritional content of the food based on the food items and the food portions in the image of the food.

13. The system of claim 9, wherein the operations comprise:
receiving, by the computing device, training data that includes nutritional content of consumed food, blood glucose levels of users before consuming the consumed food, blood glucose levels of the users after consuming the consumed food, and insulin dosages taken upon consuming the consumed food; and
using machine learning, training, by the computing device, the medication recommendation model using the nutritional content of the consumed food, the blood glucose levels of the users before consuming the consumed food, the blood glucose levels of the users after eating the confused food, and the insulin dosages taken upon consuming the consumed food.

14. The system of claim 9, wherein the operations comprise:
providing, by the computing device, the data indicating the nutritional content of the food, the blood glucose level of the user, and the insulin dosage as an input to a physical activities recommendation model that is configured to generate a suggested physical activity for the user; and
providing, by the computing device for output, data indicating the suggested physical activity for the user.

15. The system of claim 14, wherein the operations comprise:
receiving, by the computing device, training data that includes nutritional content of consumed food, blood glucose levels of users before consuming the consumed food, blood glucose levels of the users after consuming the consumed food, insulin dosages taken by the users upon consuming the consumed food, and physical activities of the users; and
using machine learning, training, by the computing device, the physical activities recommendation model using the nutritional content of the consumed food, the blood glucose levels of the users before consuming the consumed food, the blood glucose levels of the users after consuming the consumed food, the insulin dosages taken by the users upon consuming the consumed food, and the physical activities of the users.

16. The system of claim 9, wherein the operations comprise:
receiving, by the computing device, an additional blood glucose level of the user;
receiving, by the computing device, data indicating that the user consumed the food and the user took the insulin dosage; and
using machine learning, updating, by the computing device, the food classification model using the additional blood glucose level of the user, the data indicating that the user consumed the food, and the data indicating that the user took the insulin dosage.

17. A non-transitory computer-readable medium storing software comprising instructions executable by one or more computers which, upon such execution, cause the one or more computers to perform operations comprising:
receiving, by a computing device, an image of food and a blood glucose level of a user;
providing, by the computing device, the image of the food as an input to a food classification model that is configured to generate data indicating nutritional content of the food based on the image of the food;
receiving, by the computing device and from the food classification model, the data indicating the nutritional content of the food;
providing, by the computing device, the data indicating the nutritional content of the food and the blood glucose level of the user as an input to a medication recommendation model that is configured to generate data indicating an insulin dosage;
receiving, by the computing device and from the medication recommendation model, the data indicating the insulin dosage; and
providing, for output by the computing device, instructions for the user to take the insulin dosage upon consuming the food.

18. The medium of claim 17, wherein the operations comprise:
receiving, by the computing device, training data that includes images of food and data indicating food portions and food items included in each image of food; and
using machine learning, training, by the computing device, a food identification model using the images of food and the data indicating the food portions and the food items included in each image of food.

19. The medium of claim 17, wherein the operations comprise:
receiving, by the computing device, training data that includes nutritional content of consumed food, blood glucose levels of users before consuming the consumed food, blood glucose levels of the users after consuming the consumed food, and insulin dosages taken upon consuming the consumed food; and
using machine learning, training, by the computing device, the medication recommendation model using the nutritional content of the consumed food, the blood glucose levels of the users before consuming the consumed food, the blood glucose levels of the users after eating the confused food, and the insulin dosages taken upon consuming the consumed food.

20. The medium of claim 17, wherein the operations comprise:
receiving, by the computing device, an additional blood glucose level of the user;
receiving, by the computing device, data indicating that the user consumed the food and the user took the insulin dosage; and
using machine learning, updating, by the computing device, the food classification model using the additional blood glucose level of the user, the data indicating that the user consumed the food, and the data indicating that the user took the insulin dosage.
